(19) United States
(12) Patent Application Publication
(10) Pub. No.: US 2018/0177463 A1
(43) Pub. Date: Jun. 28, 2018

(54) SYSTEMS AND METHODS FOR ANALYZING A RESPIRATORY PARAMETER

(71) Applicant: Covidien LP, Mansfield, MA (US)

(72) Inventors: Paul S. Addison, Edinburgh (GB); James Wolstencroft, Craigellachie (GB); James Watson, Dunfermline (GB)

(21) Appl. No.: 15/903,009

(22) Filed: Feb. 22, 2018

Related U.S. Application Data
(63) Continuation of application No. 14/730,697, filed on Jun. 4, 2015.
(60) Provisional application No. 62/008,646, filed on Jun. 6, 2014.

(51) Int. Cl. A61B 5/00; A61B 5/0295; A61B 5/08; A61B 5/0205; A61B 5/113; A61B 5/083; A61B 5/1455

(52) U.S. Cl. CPC A61B 5/7275; A61B 5/0295; A61B 5/0816; A61B 5/742; A61B 5/7278; A61B 5/743; A61B 5/14551; A61B 5/0205; A61B 5/113; A61B 5/746; A61B 5/7221; A61B 5/0823; A61B 5/0836; A61B 5/7207

(57) ABSTRACT

Methods and systems are provided that determine whether a patient is breathing irregularly. A system may receive a physiological signal, such as a plethysmographic signal or an end-tidal carbon dioxide signal, from a sensor. The system may analyze the signal for one or more features indicative of irregular breathing, which may be a result of a patient talking, moving, yawning, coughing, sneezing, or the like. The system may also be configured to provide an indication of the irregular breathing.

### CROSS-REFERENCE TO RELATED APPLICATIONS

This application claims priority to U.S. patent application Ser. No. 14/730,697, filed Jun. 4, 2015, which claims priority to U.S. Provisional Application No. 62/008,646, filed Jun. 6, 2014, the entire contents of which are hereby expressly incorporated by reference for all purposes.

### BACKGROUND

The present disclosure relates generally to techniques for monitoring physiological parameters of a patient and, more particularly, to techniques for determining a respiration rate of a patient.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of systems and devices have been developed for monitoring many of these physiological characteristics. Generally, these patient monitoring systems provide doctors and other healthcare personnel with the information they need to provide the best possible healthcare for their patients. Consequently, such monitoring systems have become an indispensable part of modern medicine.

In some cases, clinicians may wish to monitor a patient's respiration rate. Respiration rate may be assessed using a wide variety of monitoring devices. For example, respiration rate may be monitored non-invasively via capnography using a carbon dioxide sensor. Additionally, respiration rate may be monitored non-invasively via photoplethysmography using a pulse oximetry sensor. However, signals obtained by the carbon dioxide sensor and/or by the pulse oximetry sensor may be adversely affected by certain events, such as the patient talking, moving, yawning, coughing, or the like. Thus, the signals may not always accurately reflect the patient's respiration rate.

### BRIEF DESCRIPTION OF THE DRAWINGS

Advantages of the disclosed techniques may become apparent upon reading the following detailed description and upon reference to the drawings in which:

FIG. 1 is a schematic drawing of a system including a patient monitor and a capnograph, in accordance with an embodiment;

FIG. 2 is a block diagram of the patient monitor of FIG. 1, in accordance with an embodiment;

FIG. 3 is a block diagram of the capnograph of FIG. 1 coupled to a patient, in accordance with an embodiment;

FIG. 4A illustrates a plot of a plethysmographic waveform generated using the patient monitor of FIG. 1, in accordance with an embodiment;

FIG. 4B illustrates a plot of a carbon dioxide waveform generated using the capnograph of FIG. 1, in accordance with an embodiment;

FIG. 5 is a flow diagram of a method for providing an indication of irregular breathing using the system of FIG. 1, in accordance with an embodiment;

FIG. 6 is a flow diagram of a method for providing an indication of a cause of irregular breathing using the system of FIG. 1, in accordance with an embodiment;

FIG. 7 is an illustration of a display including an indication of irregular breathing, in accordance with an embodiment; and

FIG. 8 is an illustration of a display including a waveform with portions corresponding to irregular breathing removed, in accordance with an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As noted above, clinicians may wish to monitor a patient's respiration rate. Respiration rate may be determined using a wide variety of medical monitoring techniques, such as, for example, capnography and/or photoplethysmography. However, signals acquired using capnography and/or photoplethysmography may be adversely affected by certain events, such as a patient talking, moving, coughing, sneezing, yawning, or the like, which may result in artifacts or noise in the signals. For example, in some embodiments, respiration rate may be determined based at least in part upon modulations in a waveform (e.g., a plethysmographic waveform, an end-tidal carbon dioxide waveform, or any other suitable waveform), and the presence of certain events, such as talking, motion, coughing, sneezing, yawning, or the like, may alter the modulations in the waveform. As such, a calculated respiration rate may be adversely affected during such events. In particular, portions of a respiration waveform corresponding to such events may not contain clinically useful information for calculating respiration rate. However, it may be difficult for a caregiver to identify such events from the calculated respiration rate and/or the displayed respiration waveform.

Accordingly, the present embodiments provide techniques for detecting events that may adversely affect the calculated respiration rate and for alerting the caregiver of such events. For example, a monitor may be configured to analyze a waveform (e.g., a plethysmographic waveform, an end-tidal carbon dioxide waveform, or any other suitable waveform) for one or more features (e.g., characteristics of the waveform) indicative of the presence of events that may affect the determination of respiration rate (e.g., talking, motion, body movement, coughing, sneezing, yawning, or the like). As used herein, motion may include any action that cause a change in position of at least a portion of a patient's body and may include talking, body movement, coughing, sneezing, yawning, or the like. Additionally, as used herein, body movement may include abduction, adduction, extension, flexion, rotation, and/or circumduction of any portion of a patient's body. In certain embodiments, the one or more features indicative of the presence of events that may affect the determination of respiration rate may include a spread (e.g., variation) in the distribution of breath periods of the waveform, a ratio of the inhalation periods of the waveform to the exhalation periods of the waveform, and/or irregularity (e.g., asymmetry) of the peaks of the waveform. Additionally, in certain embodiments, the monitor may be configured to provide one or more indications of the presence of events that may affect the determination of respiration rate and/or more remove portions of the waveform corresponding to such events for the determination of respiration rate.

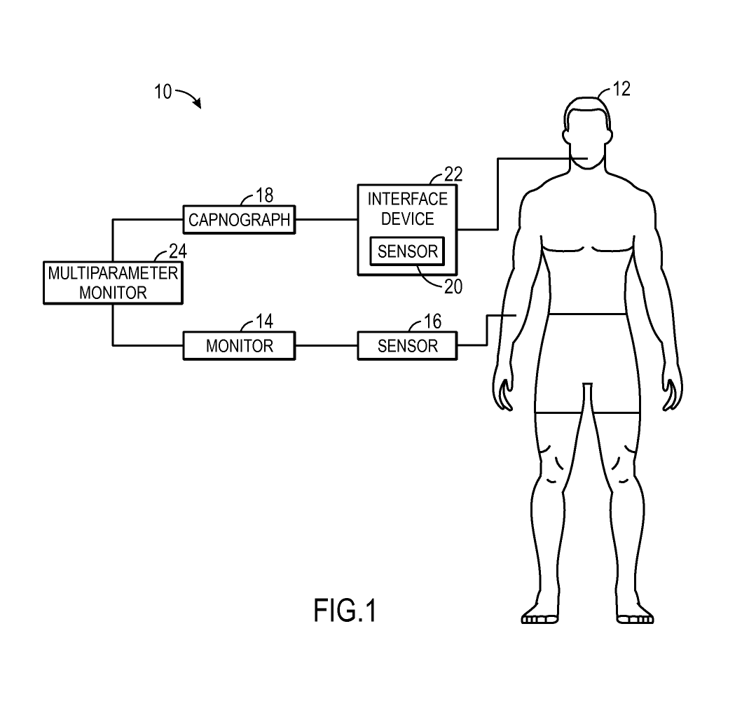

With the foregoing in mind, FIG. 1 illustrates a schematic diagram of a system 10 for implementing techniques for monitoring physiological parameters of a patient 12, such as respiration. The system 10 may include a patient monitor 14 operatively coupled to one or more plethysmographic sensors 16. The one or more plethysmographic sensors 16 may be pulse oximetry sensors or any other suitable sensors. The plethysmographic sensors 16 may be configured to generate physiological signals, which may include a plethysmographic waveform, a pulse oximetry signal, or any other signal corresponding to blood flow in the patient 12. As will be described in more detail below, the patient monitor 14 may be configured to determine physiological characteristics of the patient 12 based on the generated physiological signals, such as, for example, respiration rate, respiratory effort, blood oxygen saturation, heart rate, or the like. The patient monitor 14 may be a pulse oximeter monitor, such as those available from Covidien LP, or any other suitable monitor, such as a vital signals monitor, a critical care monitor, an obstetrical care monitor, or the like.

In certain embodiments, the system 10 may be configured to implement capnography techniques for determining physiological parameters (e.g., respiration rate) of the patient 12. For example, the system 10 may include a capnograph 18 operatively coupled to one or more carbon dioxide sensors 20. As will be described in more detail below, the capnograph 18 may be configured to determine physiological characteristics of the patient 12 using signals generated from the carbon dioxide sensor 20, such as, for example, end tidal carbon dioxide concentration, respiration rate, respiratory effort, or the like. The carbon dioxide sensor 20 may be any suitable sensor for measuring carbon dioxide in respiratory gases or the tissue of the patient 12. For example, the carbon dioxide sensor 20 may include chemical, electrical, optical, non-optical, quantum-restricted, electrochemical, enzymatic, spectrophotometric, fluorescent, or chemiluminescent indicators or transducers. In embodiments in which the carbon dioxide sensor 20 is configured to measure carbon dioxide in respiratory gases of the patient 12, the carbon dioxide sensor 20 may be disposed within, integrated with, or generally coupled to an interface device 22. The interface device 22 may be any suitable device for collecting respiratory gases of the patient 12, such as a breathing mask (e.g., a nasal mask, a nasal/oral mask, a nasal prong, a full-face mask, or the like). In some embodiments, the interface device 22 may be a nebulizer, tracheostomy tube, or an endotracheal tube. In certain embodiments, the interface device 22 may be coupled to a ventilator or other device configured to support or supplement the respiratory efforts of the patient 12.

In certain embodiments, the system 10 may also include a multi-parameter monitor 24 operatively coupled to the patient monitor 14 and/or the capnograph 18. In addition to the patient monitor 14 and/or the capnograph 18, or alternatively, the multi-parameter monitor 24 may be configured to calculate physiological characteristics of the patient 12. That is, in some embodiments, the multi-parameter monitor 24 may be configured to receive signals from the plethysmographic sensor 16 and/or signals from the carbon dioxide sensor 20 and may calculate respiration rate using signals from the plethysmographic sensor 16, signals from the carbon dioxide sensor 20, or both. Additionally, the multi-parameter monitor 24 may provide a central display for information from the patient monitor 14, the capnograph 18, and/or other medical monitoring devices or systems. For example, the multi-parameter monitor 24 may display a plethysmographic waveform from the patient monitor 14, an end tidal carbon dioxide concentration waveform from the capnograph 18, and/or the patient's respiration rate from the patient monitor 14 and/or the capnograph 18. In one embodiment, the multi-parameter monitor 24 may be configured to analyze the values of the respiration rate received from the patient monitor 14 and the capnograph 18 and may determine which value of the respiration rate to display (e.g., which value is determined to be more accurate). In other embodiments, the multi-parameter monitor 24 may be configured to average the values of the respiration rate received from the patient monitor 14 and the capnograph 18 and may display the averaged respiration rate. Additionally, the multi-parameter monitor 24 may indicate an alarm condition via a display and/or a speaker if the patient's physiological characteristics are determined to be outside of an expected threshold or range. In certain embodiments, the multi-parameter monitor 24, the patient monitor 14, and/or the capnograph 18 may be connected to a network to enable the sharing of information with servers or other workstations.

While the illustrated embodiment of the system 10 includes components for implementing photoplethysmography techniques (e.g., the patient monitor 14 and the plethysmographic sensor 16) and components for implementing capnography techniques (e.g., the capnograph 18 and the carbon dioxide sensor 20), it should be noted that, in certain embodiments, the system 10 may not include the patient monitor 14 and the plethysmographic sensor 16 and/or may not include the capnograph 18 and the carbon dioxide sensor 20. That is, in some embodiments, the present techniques for determining respiration rate and/or determining whether the patient 12 is breathing irregularly may be implemented by the patient monitor 14 using signals from the plethysmographic sensor 16, without the use of the capnograph 18. Further, in other embodiments, the present techniques for determining respiration rate and/or determining whether the patient 12 is breathing irregularly may be implemented by the capnograph 18 using signals from the carbon dioxide sensor 20, without the use of the patient monitor 14. Additionally, in other embodiments, the present techniques for determining respiration rate and/or determining whether the patient 12 is breathing irregularly may be implemented by the multi-parameter monitor 24, or any other suitable processor-based device, using signals from the plethysmographic sensor 16, signals from the carbon dioxide sensor 20, or signals from both, without the use of the patient monitor 14 or the capnograph 18. In some embodiments, the system 10 may additionally or alternatively include technologies configured to determine respiration rate and/or to detect events that may adversely affect the determination of respiration rate (e.g., talking, coughing, motion, body movement, sneezing, yawning, or the like) using any suitable signal. By way of example, suitable signals may include trans-thoracic impedance (TTI) signals, electrocardiography (ECG) signals, arterial line signals, blood flow signals, ultrasound signals, airflow signals, humidity signals, microphone signals, bed pressure sensor signals, accelerometer signals, remote sensing signals (e.g., video, infrared, radar, etc.), thoracic volume signals (e.g., from a chest band), and/or temperature signals (e.g., from a nasal thermistor). Accordingly, the system 10 may include any other suitable sensor, monitor, medical device, or any combinations thereof for acquiring signals for the determination of respiration rate and/or detecting events that may adversely affect the determination of respiration rate.

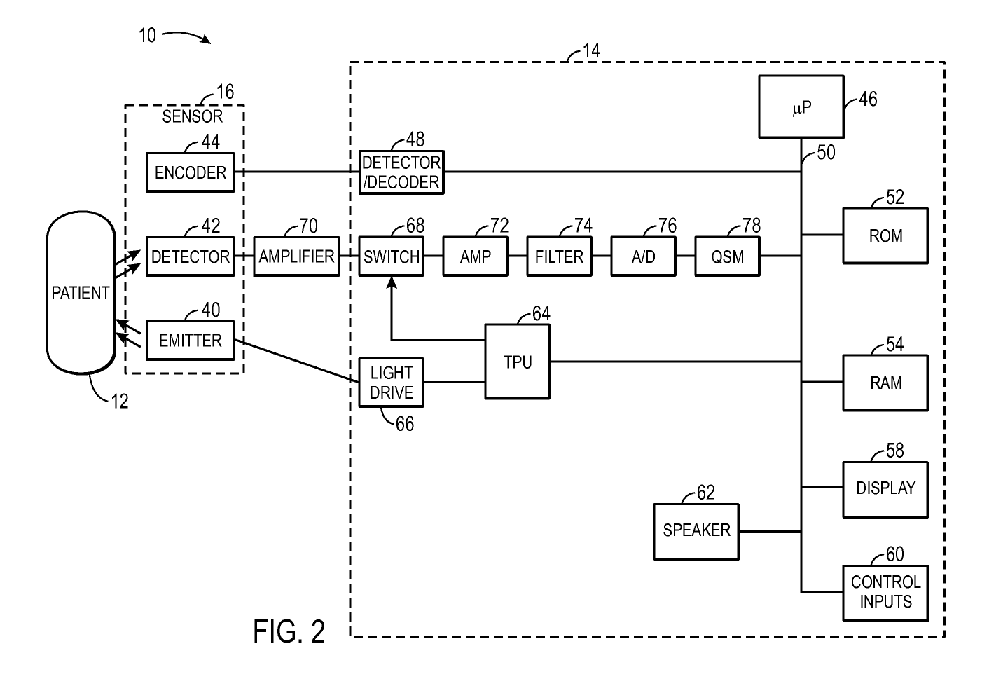

Turning to FIG. 2, a simplified block diagram of the patient monitor 14 and the plethysmographic sensor 16 of the system 10 is illustrated in accordance with an embodiment. As provided herein, the plethysmographic sensor 16 may be a sensor suitable for detection of one or more physiological parameters. The plethysmographic sensor 16 may include optical components, such as one or more emitters 40 and one or more detectors 42. In one embodiment, the sensor 16 may be configured for photo-electric detection of blood and tissue constituents. For example, the plethysmographic sensor 16 may include pulse oximetry sensing functionality for determining the oxygen saturation of blood as well as other parameters (e.g., respiration rate, arrhythmia detection) from the plethysmographic waveform detected by the oximetry technique. An oximetry system may include a light sensor (e.g., the plethysmographic sensor 16) that is placed at a site on a patient, typically a fingertip, toe, forehead or earlobe, or in the case of a neonate, across a foot. The plethysmographic sensor 16 may pass light using the emitter 40 through blood perfused tissue and photoelectrically sense the absorption of light in the tissue. For example, the patient monitor 14 may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (photoplethysmography) signal. The light intensity or the amount of light absorbed may then be used to calculate the amount of the blood constituent (e.g., oxyhemoglobin) being measured and other physiological parameters such as the pulse rate and when each individual pulse occurs. Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. At least two, e.g., red and infrared (IR), wavelengths may be used because it has been observed that highly oxygenated blood will absorb relatively less red light and more infrared light than blood with a lower oxygen saturation. However, it should be understood that any appropriate wavelengths, e.g., green, etc., may be used as appropriate. Further, photoplethysmography measurements may be determined based on one, two, or three or more wavelengths of light.

The emitter 40 and the detector 42 may be arranged in a reflectance or transmission-type configuration with respect to one another. However, in embodiments in which the plethysmographic sensor 16 is configured for use on a patient's forehead (e.g. either alone or in conjunction with a hat or headband), the emitters 40 and detectors 42 may be in a reflectance configuration. The emitter 40 may also be a light emitting diode, superluminescent light emitting diode, a laser diode or a vertical cavity surface emitting laser (VCSEL). The emitter 40 and the detector 42 may also include optical fiber sensing elements. The emitter 40 may include a broadband or “white light” source, in which case the detector 42 could include any of a variety of elements for selecting specific wavelengths, such as reflective or refractive elements, absorptive filters, dielectric stack filters, or interferometers. These kinds of emitters and/or detectors would typically be coupled to the plethysmographic sensor 16 via fiber optics.

In certain embodiments, the emitter 40 and detector 42 may be configured for pulse oximetry. It should be noted that the emitter 40 may be capable of emitting at least two wavelengths of light, e.g., red and infrared (IR) light, into the tissue of a patient, where the red wavelength may be between about 600 nanometers (nm) and about 700 nm, and the IR wavelength may be between about 800 nm and about 1000 nm. The emitter 40 may include a single emitting device, for example, with two LEDs, or the emitter 40 may include a plurality of emitting devices with, for example, multiple LEDs at various locations. In some embodiments, the LEDs of the emitter 40 may emit three or more different wavelengths of light. Regardless of the number of emitting devices, light from the emitter 40 may be used to measure, as provided herein, a physiological parameter, such as a pulse rate, oxygen saturation, respiration rate, respiration effort, continuous non-invasive blood pressure, cardiac output, fluid responsiveness, perfusion, pulse rhythm type, hydration level, or any combination thereof. In certain embodiments, the sensor measurements may also be used for determining water fraction, hematocrit, or other physiologic parameters of the patient. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In any suitable configuration of the plethysmographic sensor 16, the detector 42 may be an array of detector elements that may be capable of detecting light at various intensities and wavelengths. The detector 42 may convert the received light at a given intensity, which may be directly related to the absorbance and/or reflectance of light in the tissue of the patient 12, into an electrical signal. That is, when more light at a certain wavelength is absorbed, less light of that wavelength is typically received from the tissue by the detector 42, and when more light at a certain wavelength is reflected, more light of that wavelength is typically received from the tissue by the detector 42. The detector 42 may receive light that has not entered the tissue to be used as a reference signal. After converting the received light to an electrical signal, the detector 42 may send the signal to the patient monitor 14, where physiological characteristics may be calculated based at least in part on the absorption and/or reflection of light by the tissue of the patient.

In certain embodiments, the plethysmographic sensor 16 may also include an encoder 44 that may provide signals indicative of the wavelength of one or more light sources of the emitter 40, which may allow for selection of appropriate calibration coefficients for calculating a physical parameter such as blood oxygen saturation or respiration rate. The encoder 44 may, for instance, be a coded resistor, EEPROM or other coding devices (such as a capacitor, inductor, PROM, RFID, parallel resident currents, or a colorimetric indicator) that may provide a signal to a processor 46 of the patient monitor 14 related to the characteristics of the plethysmographic sensor 16 to enable the processor 46 to determine the appropriate calibration characteristics of the plethysmographic sensor 16. In some embodiments, the encoder 44 and/or the detector/decoder 48 may not be present.

Signals from the detector 42 and/or the encoder 44 may be transmitted to the patient monitor 14. The patient monitor 14 may include one or more processors 46 coupled to an internal bus 50. Also connected to the bus 50 may be a ROM memory 52, a RAM memory 54, a display 58, control inputs 60, and a speaker 62. A time processing unit (TPU) 64 may provide timing control signals to light drive circuitry 66, which may control when the emitter 40 is activated, and if multiple light sources are used, the multiplexed timing for the different light sources. The TPU 64 may also control the gating-in of signals from detector 42 through a switching circuit 68. These signals are sampled at the proper time, depending at least in part upon which of multiple light sources is activated, if multiple light sources are used. The received signal from the detector 42 may be passed through one or more amplifiers (e.g., amplifiers 70 and 72), a low pass filter 74, and an analog-to-digital converter 76 for amplifying, filtering, and digitizing the electrical signals from the plethysmographic sensor 16. The digital data may then be stored in a queued serial module (QSM) 78, for later downloading to RAM 54 as QSM 78 fills up. In an embodiment, there may be multiple parallel paths for separate amplifiers, filters, and A/D converters for multiple light wavelengths or spectra received.

Based at least in part upon the received signals corresponding to the light received by optical components of the plethysmographic sensor 16, the processor 46 may calculate oxygen saturation, respiration rate, and/or heart rate using various algorithms. It should be noted that, in order to measure respiration rate, embodiments of the present disclosure may utilize systems and methods such as those disclosed in U.S. Pat. No. 7,035,679, filed Jun. 21, 2002, U.S. Pat. No. 8,255,029, filed Feb. 27, 2004, and U.S. Publication Application No. 2013/0079606, filed Sep. 23, 2011, which are each incorporated herein by reference in their entirety for all purposes. In addition, the processor 46 may detect events (e.g., artifacts or noise in the plethysmographic waveform) that may adversely affect the determination of respiration rate, such as talking, motion, body movement, coughing, sneezing, yawning, or the like, and may display one or more indications of such events and/or remove the artifacts for the determination of respiration rates using various methods, such as those provided herein. These algorithms may employ certain coefficients, which may be empirically determined, and may correspond to the wavelengths of light used. The algorithms and coefficients may be stored in the ROM 52 or other suitable computer-readable storage medium and accessed and operated according to processor 46 instructions.

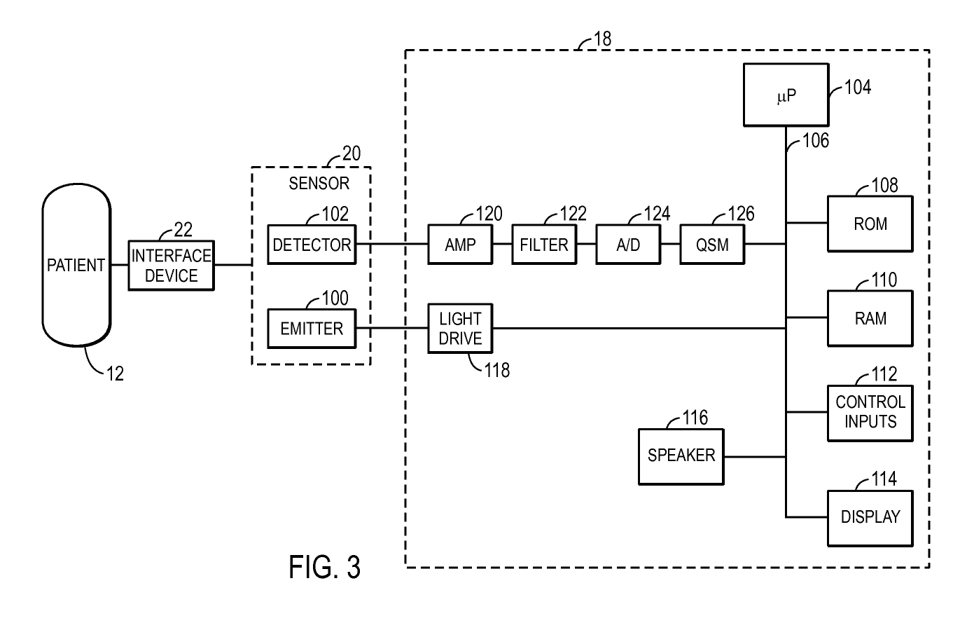

As noted above, the system 10 may also include components for implementing capnography techniques (e.g., the capnograph 18 and the carbon dioxide sensor 20) to acquire signals for determining respiration rate and/or for detecting events that may adversely affect the determination of respiration rate. For example, FIG. 3 illustrates a simplified block diagram of the capnograph 18 and the carbon dioxide sensor 20 of the system 10. The carbon dioxide sensor 20 may include any appropriate sensor or sensor element for assessing expired carbon dioxide, including chemical, electrical optical, non-optical, quantum-restricted, electrochemical, enzymatic, spectrophotometric, fluorescent, or chemiluminescent indicators or transducers. Generally, the carbon dioxide sensor 20 may include any indicator that is sensitive to the presence of carbon dioxide and that is capable of being calibrated to give a response signal corresponding to a given predetermined concentration of carbon dioxide. In certain embodiments, the carbon dioxide sensor 20 may monitor the partial pressure or concentration of carbon dioxide in the respiratory gases. By monitoring the carbon dioxide changes during the breath cycle, the number of breaths per minute (i.e., the respiration rate) may be determined.

In certain embodiments, the carbon dioxide sensor 20 may include optical components, such as an emitter 100 and a detector 102. For example, the emitter 100 may be one or more light emitting diodes adapted to transmit one or more wavelengths of light in the red to infrared range, and the detector 102 may be one or more photodetectors selected to receive light in the range or ranges emitted from the emitter 100. Alternatively, the emitter 100 may also be a laser diode or a vertical cavity surface emitting laser (VCSEL). The emitter 100 and detector 102 may also include optical fiber sensing components. The emitter 100 may include a broadband or “white light” source, in which case the detector 102 could include any of a variety of elements for selecting specific wavelengths, for example, reflective or refractive elements or interferometers. These kinds of emitters 100 and/or detectors 102 would typically be coupled to the rigid or rigidified sensor 20 via fiber optics. Alternatively, the carbon dioxide sensor 20 may sense light detected through the respiratory gas at a different wavelength from the light emitted into the respiratory gas. Such sensors may be adapted to sense fluorescence, phosphorescence, Raman scattering, Rayleigh scattering and multi-photon events or photoacoustic effects. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the ultrasound, radio, microwave, millimeter wave, infrared, visible, ultraviolet, gamma ray or X-ray spectra.

The emitter 100 and the detector 102 may be arranged in a reflectance or transmission-type configuration with respect to one another. For example, in embodiments in which the carbon dioxide sensor 20 is integrated with the interface device 22 (e.g., embedded within a wall of the interface device 22), the emitter 100 and the detector 102 may be arranged in a reflectance-type configuration. Alternatively, in embodiments in which the carbon dioxide sensor 20 is disposed about the interface device 22 (e.g., surrounding a portion of tubing of the interface device 22), the emitter 100 and the detector 102 may be arranged in a transmission-type configuration.

Signals from the detector 102 may be transmitted to the capnograph 18. The capnograph 18 may include one or more processors 104 coupled to an internal bus 106. Also connected to the bus 106 may be a ROM memory 108, a RAM memory 110, control inputs 112, a display 114, and a speaker 116. Light drive circuitry 118 may control when the emitter 100 is activated. The received signal from the detector 102 may be passed through one or more amplifiers (e.g., amplifier 120), a filter 122, and an analog-to-digital converter 124 for amplifying, filtering, and digitizing the electrical signals from the carbon dioxide sensor 20. The digital data may then be stored in a queued serial module (QSM) 126 for later downloading to the RAM 110 as the QSM 126 fills up. In one embodiment, there may be multiple parallel paths for separate amplifiers, filters, and A/D converters for multiple light wavelengths or data received.

Based at least in part upon the received signals from the carbon dioxide sensor 20, the processor 104 may calculate the partial pressure of carbon dioxide in the inhaled and/or exhaled breaths, the concentration of carbon dioxide in the inhaled and/or exhaled breaths, end tidal carbon dioxide, respiration rate, expiratory pH, and/or any other suitable parameters using various algorithms. In addition, the processor 104 may detect events (e.g., artifacts or noise in the carbon dioxide waveform) that may adversely affect the determination of respiration rate, such as talking, motion, body movement, coughing, sneezing, yawning, or the like, and may display one or more indications of such events and/or remove the artifacts for the determination or respiration rates using various methods, such as those provided herein. These algorithms may employ certain coefficients, which may be empirically determined, and may correspond to the wavelengths of light used. The algorithms and coefficients may be stored in the ROM 108 or other suitable computer-readable storage medium and accessed and operated according to processor 104 instructions.

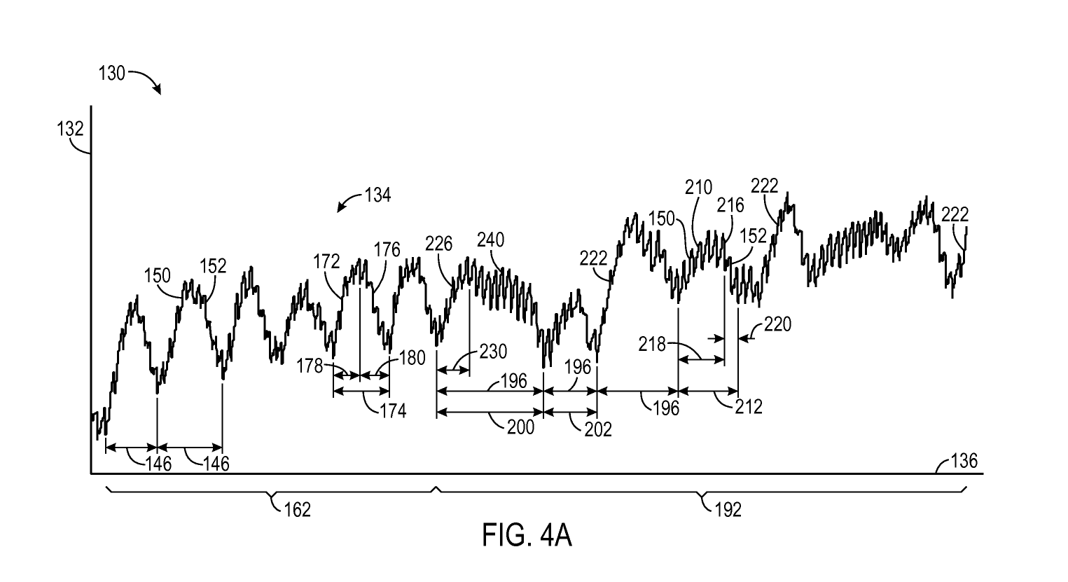

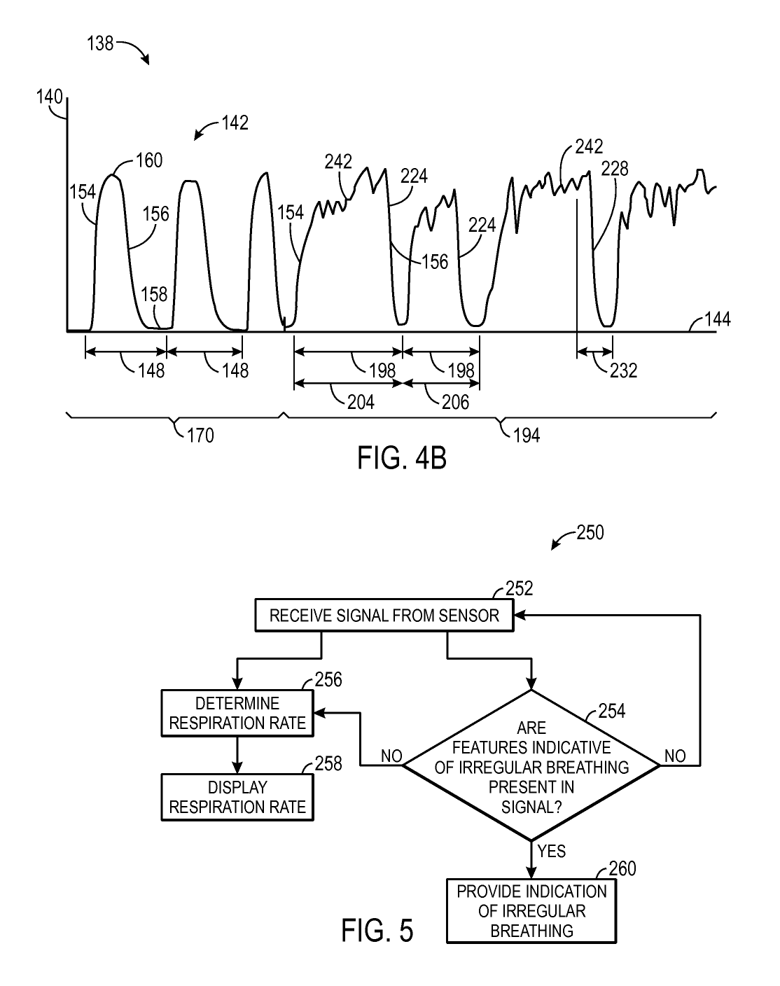

FIG. 4 illustrates a plethysmographic waveform and a carbon dioxide waveform that may be displayed and/or analyzed by the patient monitor 14, the capnograph 18, the multi-parameter monitor 24, or any other suitable processor-based device. As noted above, in some embodiments, the plethysmographic waveform and/or the carbon dioxide waveform may be analyzed by only one processor-based device (e.g., the patient monitor 14, the capnograph 18, or the multi-parameter monitor 24), using the techniques as described below, to determine respiration rate and to determine whether the patient 12 is breathing irregularly. For example, in one embodiment, the patient monitor 14 may receive signals from both the plethysmographic sensor 16 and the carbon dioxide sensor 20 and may determine whether the patient 14 is breathing irregularly based on the signals from both the plethysmographic sensor 16 and the carbon dioxide sensor 20. Further, in another embodiment, the capnograph 18 may receive signals from both the plethysmographic sensor 16 and the carbon dioxide sensor 20 and may determine whether the patient 14 is breathing irregularly based on the signals from both the plethysmographic sensor 16 and the carbon dioxide sensor 20. Additionally, in another embodiment, the multi-parameter monitor 24 may receive signals from both the plethysmographic sensor 16 and the carbon dioxide sensor 20 and may determine whether the patient 14 is breathing irregularly based on the signals from both the plethysmographic sensor 16 and the carbon dioxide sensor 20.

In particular, FIG. 4A illustrates a first plot 130, which shows the amplitude (on y-axis 132) of an example plethysmographic waveform 134 over time (x-axis 136), and FIG. 4B illustrates a second plot 138, which shows the amplitude (on y-axis 140) of an example carbon dioxide waveform 142 over time (x-axis 144). The plethysmographic waveform 134 may be generated by the plethysmographic sensor 16 and analyzed by the processor 46 to determine respiration rate. Additionally, the carbon dioxide waveform 142 may be generated by the carbon dioxide sensor 20 and analyzed by the processor 104 to determine respiration rate. Further, the processor 46 and the processor 104 may analyze the plethysmographic waveform 134 and the carbon dioxide waveform 142, respectively, for one or more features that may be indicative of irregular breathing, which may be caused by one or more events, such as talking, motion, coughing, sneezing, yawning, or the like. Additionally, in certain embodiments, the processor 46 and/or the processor 104 may be configured to identify such events (e.g., talking, motion, body movement, coughing, sneezing, yawning, or the like) based at least in part upon the detection of the one or more features.

The plethysmographic waveform 134 and the carbon dioxide waveform 142 each generally rise and fall over the course of a breath period (e.g., breath periods 146 of the plethysmographic waveform 134 and breath periods 148 of the carbon dioxide waveform 142). In particular, the amplitude of the plethysmographic waveform 134 increases (e.g., rises) during inhalation and an inspiratory upstroke 150 is observed. During exhalation, the amplitude of the plethysmographic waveform 134 decreases (e.g., falls) and an expiratory downstroke 152 is observed. In contrast, the amplitude of the carbon dioxide waveform 142 increases during exhalation and decreases during inhalation. In particular, the carbon dioxide waveform 142 includes expiratory upstrokes 154 and inspiratory downstrokes 156. More specifically, the carbon dioxide waveform 142 may include an inspiratory baseline 158 that is indicative of inspired gas, which is generally devoid of or includes a minimal amount of carbon dioxide. The inspiratory baseline 158 is followed by the expiratory upstroke 154. The carbon dioxide waveform 142 may include an alveolar plateau 160 between the expiratory upstroke 154 and the inspiratory downstroke 156.

As illustrated, the plethysmographic waveform 134 and the carbon dioxide waveform 142 each include a first portion (e.g., a first portion 162 of the plethysmographic waveform 134 and a first portion 170 of the carbon dioxide waveform 142) that may be indicative of regular (e.g., normal) breathing. Specifically, periods of regular breathing may be periods when the patient 12 is not talking, moving, coughing, sneezing, yawning, or the like. Periods of regular breathing may provide clinically useful information for the calculation of respiration rate and, in particular, may provide a more accurate calculation of respiration rate as compared to periods when the patient is 12 is not talking, moving, coughing, sneezing, yawning, or the like.

The first portion 162 of the plethysmographic waveform 134 and the first portion 170 of the carbon dioxide waveform 142 may each include generally periodic breath periods. In particular, the spread (e.g., variance, standard deviation) of a distribution of the breath periods 146 and 148 in the first portion 162 and the first portion 170, respectively, may be less than a predetermined threshold for the spread of the breath distribution. That is, the patient 12 may inhale and exhale with a generally constant frequency. In certain embodiments, the predetermined threshold for the spread of the breath distribution may be based at least in part upon a mean respiration rate of the patient 12. Accordingly, in certain embodiments, the processor 46 and the processor 104 may be configured to analyze the plethysmographic waveform 134 and the carbon dioxide waveform 142, respectively, for one or more features indicative of normal breathing (e.g., generally periodic breath periods) and may be configured to determine that the patient 12 is breathing normally (e.g., not talking, moving, coughing, yawning, sneezing, etc.) based at least in part upon one or more detected features indicative of normal breathing and/or based at least in part upon a determination that the spread of the breath periods is less than a predetermined threshold.

Additionally, each breath period 146 in the first portion 162 of the plethysmographic waveform 134 may be generally symmetrical. That is, the inspiratory upstroke 150 of each breath period 146 of the first portion 162 may have a generally similar duration and slope (e.g., absolute value of the slope) to that of the respective expiratory downstroke 152. For example, the slope 172 of the inspiratory upstroke 150 for a breath period 174 of the first portion 162 may be within a predetermined range of an absolute value of the slope 176 of the expiratory downstroke 152 for the same breath period 174. Additionally, the period 178 (e.g., duration) of the inspiratory upstroke 150 may be within a predetermined range of the period 180 of the expiratory downstroke 152. Accordingly, in certain embodiments, the processor 46 may be configured to analyze the plethysmographic waveform 134 for generally symmetrical breath periods and may be configured to determine that the patient 12 is breathing normally (e.g., not talking, moving, coughing, yawning, sneezing, etc.) based at least in part upon the determination that plethysmographic waveform 134 includes generally symmetrical breath periods.

Additionally or alternatively, the processor 46 and the processor 104 may be configured to analyze the plethysmographic waveform 134 and the carbon dioxide waveform 142, respectively, for one or more features indicative of irregular breathing, such as talking, motion, body movement, coughing, sneezing, yawning, or the like. As will be described in more detail below, talking, motion, body movement, coughing, sneezing, and/or yawning, may result in irregular periodicity of breath periods, asymmetric breath periods, short inhalations relative to exhalations, sharp inhalations (e.g., steep inspiratory upstrokes), and/or irregular peaks on the waveform. As illustrated, the plethysmographic waveform 134 and the carbon dioxide waveform 142 include a period of irregular breathing 192 and 194, respectively. The periods of irregular breathing 192 and 194 may each be indicative of talking, motion, body movement, coughing sneezing, yawning, and/or any other action that may alter the patient's breathing. The periods of irregular breathing 192 and 194 may not provide clinically useful information and/or may decrease the accuracy of the determination of respiration rate and/or other physiological parameters. Thus, it may be desirable to identify periods of irregular breathing, to provide an indication to a user of the periods of irregular breathing, and/or to exclude data during the periods of irregular breathing from the calculation of respiration rate.

In contrast to the first portions 162 and 170, the periods of irregular breathing 192 and 194 include breath periods 196 and 198, respectively, which are irregular (e.g., inconstant) over time. In particular, the spread (e.g., variance, standard deviation) of a distribution of the breath periods 196 and 198 in the period of irregular breathing 192 and 194, respectively, may be greater than a predetermined threshold. For example, as illustrated in FIG. 4A, the period of irregular breathing 192 of the plethysmographic waveform 134 includes a first breath period 200 and a second breath period 202, and the first breath period 200 is longer than the second breath period 202, which may increase the spread of the distribution of the breath periods 196. Similarly, the period of irregular breathing 194 of the carbon dioxide waveform 142 includes a first breath period 204 and a second breath period 206, and the first breath period 204 is longer than the second breath period 206.

Accordingly, the processor 46 and the processor 104 may be configured to analyze the plethysmographic waveform 134 and the carbon dioxide waveform 142, respectively, for irregular breath periods and, in some embodiments, may be configured to calculate the spread (e.g., standard deviation) of a distribution of breath periods. Further, the processor 46 and the processor 104 may be configured to determine that the patient 12 is breathing irregularly based at least in part upon the detection of irregular breath periods (e.g., irregular breath periods 196 and/or 198) and/or a determination that the spread of the distribution of breath periods (e.g., irregular breath periods 196 and/or 198) is greater than a predetermined threshold.

Additionally, one or more breath periods 196 in the period of irregular breathing 192 of the plethysmographic waveform 134 may be asymmetrical. That is, the inspiratory upstroke 150 of one or more breath periods 196 in the period of irregular breathing 192 may have a duration (e.g., period) and/or a slope (e.g., absolute value of the slope) that is substantially different from (e.g., outside of a predetermined range of) that of the respective expiratory downstroke 152. By way of example, the slope 210 of the inspiratory upstroke 150 for a breath period 212 in the period of irregular breathing 192 may be outside of a predetermined range of the slope 216 of the expiratory downstroke 152 for the same breath period 212. Additionally, the period 218 (e.g., duration) of the inspiratory upstroke 150 of the breath period 212 may be outside of a predetermined range of the period 220 of the expiratory downstroke 152 for the breath period 212.

Accordingly, in certain embodiments, the processor 46 may be configured to analyze the plethysmographic waveform 134 for asymmetrical breath periods (e.g., breath periods 196). For example, the processor 46 may be configured to compare the slope of the inspiratory upstroke of each breath period to the slope of the expiratory downstroke of the respective breath period. Additionally, the processor 46 may be configured to compare the period of the inspiratory upstroke of each breath period to the period of the expiratory downstroke of the respective breath period. Furthermore, the processor 46 may be configured to determine that the patient 12 is breathing irregularly based at least in part upon a determination that the slopes of one or more inspiratory upstrokes of one or more breath periods are outside of a predetermined range of the slopes of one or more expiratory downstrokes of the respective one or more breath periods, a determination that periods of one or more inspiratory upstrokes of one or more breath periods are outside of a predetermined range of the periods of one or more expiratory downstrokes of the respective one or more breath periods, and/or the detection of any other features indicative of asymmetric breath periods.

Furthermore, irregular breathing, and in particular, irregular breathing due to talking or yawning, may result in sharp inhalations. For example, irregular breathing may include inhalations that are short (e.g., a shorter period or duration) and/or rapid (e.g., a steeper or greater slope) relative to inhalations of normal breathing and/or relative to exhalations of the respective breath period. This may occur because the patient 12 may take a quick, deep breath before talking or in between talking (e.g., vocal pauses) and may slowly exhale over the course of the talking. For example, as illustrated in FIG. 4A, the plethysmographic waveform 134 may include one or more steep inspiratory upstrokes 222 that have a slope greater than a predetermined threshold. In some embodiments, the predetermined threshold may be based at least in part upon an average slope of the inspiratory upstrokes 150 of the first portion 162. Similarly, as illustrated in FIG. 4B, the carbon dioxide waveform 142 may include one or more steep inspiratory downstrokes 224 that have a slope greater than a predetermined threshold, which may be based at least in part upon an average slope of the inspiratory downstrokes 156 of the first portion 170. In certain embodiments, the predetermined thresholds for the slope of the inspiratory upstrokes 150 and the inspiratory downstrokes 156 may be determined based upon a predetermined deviation from the respective average slope value. Accordingly, the processor 46 and/or the processor 104 may be configured to compare the slope of the inspiratory upstrokes 150 and the slope of the inspiratory downstrokes 156, respectively, to a respective predetermined threshold. Furthermore, the processor 46 and/or the processor 104 may be configured to determine that the patient 12 is breathing irregularly based at least in part upon a determination that the slope of the inspiratory upstroke 150 is greater than a predetermined threshold and/or a determination that the slope of the inspiratory downstroke 154 is greater than a predetermined threshold.

Additionally, the plethysmographic waveform 134 and the carbon dioxide waveform 142 may include one or more short inspiratory upstrokes 226 and inspiratory downstrokes 228, respectively. For example, the short inspiratory upstroke 226 of the plethysmographic waveform 134 may have a period 230 that is less than a predetermined threshold, which may be based at least in part upon an average period of the inspiratory upstrokes of the first portion 162. Additionally, the short inspiratory downstroke 228 of the carbon dioxide waveform 142 may have a period 232 that is less than a predetermined threshold, which may be based at least in part upon an average period of the inspiratory downstroke of the first portion 170. Accordingly, the processor 46 and/or the processor 104 may be configured to compare the period of the inspiratory upstroke 150 and the inspiratory downstroke 156, respectively, to a respective predetermined threshold. Furthermore, the processor 46 and/or the processor 104 may be configured to determine that the patient 12 is breathing irregularly based at least in part upon a determination that the period of the inspiratory upstroke 150 is greater than a predetermined threshold and/or a determination that the period of the inspiratory downstroke 154 is greater than a predetermined threshold.

Additionally, irregular breathing may result in long exhalations. In particular, irregular breathing may include long exhalations relative to exhalations during normal breathing and/or relative to inhalations of the same. For example, as noted above, the patient 12 may exhale slowly over the course of talking or may exhale slowly while yawning, which may result in long exhalations. In some embodiments, the processor 46 and/or the processor 104 may be configured to calculate a ratio of the inspiratory periods to the expiratory periods for one or more breath periods. The processor 46 and/or the processor 104 may be configured to determine that the patient 12 is breathing irregularly based upon a determination that the ratio of the inspiratory periods to the expiratory periods is below a predetermined threshold. In certain embodiments, the processor 46 and/or the processor 104 may be configured to determine that the patient 12 is talking based upon a determination that the ratio of the inspiratory periods to the expiratory periods is below a predetermined threshold. Additionally, in some embodiments, the processor 46 and/or the processor 104 may be configured to characterize the variability of the ratio of the inspiratory periods to the expiratory periods over time and may be configured to determine that the patient 12 is breathing irregularly based upon a determination that the variability (e.g., the standard deviation) of the ratio is greater than a predetermined threshold.

Furthermore, the plethysmographic waveform 134 and/or the carbon dioxide waveform 142 may include features having a high variability during the periods of irregular breathing (e.g., the period of irregular breathing 192 and the period of irregular breathing 194, respectively). For example, the slope of the plethysmographic waveform 134 and the slope of the carbon dioxide waveform 142 may vary over time during the period of irregular breathing 192 and the period of irregular breathing 194, respectively. In certain embodiments, the slope of the inspiratory upstroke 150 over different breath periods 196 of the plethysmographic waveform 134 may vary over time during the period of irregular breathing 192. Additionally, the slope of the expiratory upstroke 154 over different breath periods 198 may vary over time during the period of irregular breathing 194. Accordingly, in certain embodiments, the processor 46 and the processor 104 may be configured to analyze the plethysmographic waveform 134 and the carbon dioxide waveform 142, respectively, for high variability and may be configured to determine that the patient 12 is breathing irregularly based upon the detection of high variability. For example, the processor 46 and the processor 104 may be configured to quantify the gradient of the slope of the plethysmographic waveform 134 (e.g., the slope of the inspiratory upstroke 150) and the gradient of the slope of the carbon dioxide waveform 142 (e.g., the slope of the expiratory upstroke 154), respectively. In certain embodiments, the processor 46 and/or the processor 104 may be configured to determine that the patient 12 is breathing irregularly based upon a determination that the gradient of the upstroke slope of the plethysmographic waveform 134 and/or of the carbon dioxide waveform 142, respectively, is greater than a predetermined threshold. Further, the processor 46 and/or the processor 104 may be configured to determine that the patient 12 is breathing irregularly based upon a determination that the variation (e.g., spread, standard deviation) of the gradient of the upstroke slope of the plethysmographic waveform 134 and/or of the carbon dioxide waveform 142, respectively, is greater than a predetermined threshold.

Additionally, the periods of irregular breathing 192 and 194 may include irregularity in the peak portions of the respective waveforms. For example, as illustrated in FIG. 4A, a peak portion 240 of the plethysmographic waveform 134 includes irregular peaks (e.g., ripples). Similarly, a peak portion 242 of the carbon dioxide waveform 142 may include irregular peaks. The processor 46 and/or the processor 104 may be configured to analyze the plethysmographic waveform 134 and/or the carbon dioxide waveform 142 for irregularity. In certain embodiments, the processor 46 and/or the processor 104 may be configured to quantify irregular peaks of the respective waveforms based on the number, size, and/or variability of the ripples in the peak portions 240 and 242, respectively. The processor 46 and/or the processor 104 may determine that the patient 12 is breathing irregularly based upon a determination that the value of the irregularity exceeds a predetermined threshold. In certain embodiments, the predetermined threshold may be based at least in part upon historical data for the respective waveform.

In certain embodiments, the processor 46 and/or the processor 104 may be configured to perform signal processing techniques to analyze the plethysmographic waveform 134 and/or the carbon dioxide waveform 142, respectively, to detect events such as talking, motion, coughing, sneezing, yawning, or the like. That is, rather than detecting such events by identifying features in identified breath periods, as described above, the processor 46 and/or the processor 104 may also be configured to detect the events directly from the plethysmographic waveform 134 and/or the carbon dioxide waveform 142, respectively. For example, the processor 46 and/or the processor 104 may be configured to implement various techniques, such as, for example, piecewise linear approximation, linear regression, linear combination, multivariate analysis, principal component analysis (PCA), other suitable matrix techniques, independent component analysis (ICA), linear discriminate analysis (LDA), and/or any suitable signal transform methods (e.g., fast Fourier transform (FFT), continuous wavelet transform (CWT), Hilbert transform, or Laplace transform). Furthermore, signal processing techniques may include use of neural networks (e.g., multilayer perception networks (MLP) or radial basis networks), stochastic or probabilistic classifiers (e.g., Bayesian, Hidden Markov Model (HMM), or fuzzy logic classifiers), genetic-based algorithms, propositional or predicate logics (e.g., non-monotonic or modal logics), nearest neighbor classification methods (e.g., kth nearest neighbor or learning vector quantization (LVQ) methods), or any other learning-based algorithms.

Additionally, the signal processing techniques may include the combination of the plethysmographic waveform 134 and/or the carbon dioxide waveform 142 with additional sensors, including plethysmographic sensors (e.g., the plethysmographic sensor 16), carbon dioxide sensors (e.g., the carbon dioxide sensor 20), motion sensors, pressure sensors, temperature sensors, and/or ultrasound sensors. The additional sensors may provide data to be used with the plethysmographic waveform 134 and/or the carbon dioxide waveform 142, which may aid in distinguishing physiological signals from artifacts or other non-physiological components, which may be caused by talking, motion, coughing, sneezing, yawning, or the like. Furthermore, the additional sensors may provide data to be used with the plethysmographic waveform 134 and/or the carbon dioxide waveform 142, which may aid in the identification (e.g., classification) of artifacts or other non-physiological components that may result in irregular breathing, such as talking, motion, coughing, sneezing, yawning, or the like. For example, a plethysmographic sensor (e.g., the plethysmographic sensor 16) may be configured to detect patient motion and/or to determine the state of the sensor, such as a sensor off state, which may indicate that the sensor is not properly coupled to the patient 12, and/or a disconnect state, which may indicate that the sensor is not connected to the patient monitor. In certain embodiments, in order to determine the state of the plethysmographic sensor, embodiments of the present disclosure may utilize systems and methods such as those disclosed in U.S. Pat. No. 6,035,223, filed Nov. 19, 1997, which is incorporated herein by reference in its entirety for all purposes.

With the foregoing in mind, FIG. 5 illustrates a method 250 for providing an indication of irregular breathing. The method 250 may be performed as an automated procedure by a system, such as the system 10. In addition, certain steps of the method 250 may be performed by a processor or a processor-based device, such as the patient monitor 14, the capnograph 18, and/or the multi-parameter monitor 24, which includes instructions for implementing certain steps of the method 250. As noted above, in one embodiment, the method 250 may be performed using only the patient monitor 14, the capnograph 18, the multi-parameter monitor 24, or any other suitable processor-based device. Further, the method 250 may be performed using signals from only the plethysmographic sensor 16 or using signals from only the carbon dioxide sensor 20.

The method 250 may include receiving one or more signals from one or more sensors (block 252). In certain embodiments, the one or more signals may be acquired by plethysmographic sensors (e.g., the plethysmographic sensor 16), carbon dioxide sensors (e.g., the carbon dioxide sensors 20), motion sensors, temperature sensors, pressure sensors, or any other suitable sensor. The one or more signals may include, for example, a plethysmographic waveform (e.g., the plethysmographic waveform 134), a carbon dioxide waveform (e.g., the carbon dioxide waveform 142), and/or any other suitable waveform.

The method 250 may also include determining if one or more features indicative of irregular breathing are present in the one or more waveforms of the one or more received signals (block 254). As described above, irregular breathing may result from talking, moving, coughing, sneezing, and/or yawning. In certain embodiments, detecting the one or more features indicative of irregular breathing may include detecting irregular periodicity of breath periods, asymmetric breath periods, short inhalations relative to exhalations, sharp inhalations (e.g., steep inspiratory upstrokes), and/or irregular peaks on the waveform of the received signal. In particular, the one or more features indicative of irregular breathing may be detected by analyzing the waveform using the techniques as described above with respect to FIG. 4. In some embodiments, the method 250 may include obtaining (e.g., selecting) a segment of the received signal and analyzing the segment to detect the one or more features indicative of irregular breathing. For example, the segment may correspond to data to be used to calculate respiration rate. Thus, it may be desirable to determine whether the selected segment includes features indicative of irregular breathing to determine whether to use the segment to calculate respiration rate and/or to determine whether to display a calculated respiration rate, as will be described in more detail below. In other embodiments, the method 250 may include analyzing the waveform of the signal directly using the above-described signal processing techniques.

The method 250 may also include determining respiration rate (block 256) based at least in part upon the received signal. Respiration rate may be determined using data obtained from a plethysmographic waveform 134 and/or a carbon dioxide waveform 142, as described above with respect to FIG. 2 and FIG. 3, respectively. In some embodiments, respiration rate may be determined using a segment of the signal (e.g., one or more data points of the signal). In certain embodiments, determining respiration rate (block 256) may occur in response to a determination that features indicative of irregular breathing are not present. That is, the determination that the signal or signal segment does not includes one or more features indicative of irregular breathing may indicate that the signal or signal segment includes clinically useful information that may result in an accurate calculation of respiration rate. In one embodiment, the method 250 may not determine respiration rate using a signal segment that includes one or more features indicative of irregular breathing. The determination that the signal segment includes one or more features indicative of irregular breathing may indicate that the signal segment includes one or more artifacts that may adversely affect the accuracy of the calculation of respiration rate. Thus, it may be desirable to omit signal segments including features indicative of irregular breathing from the calculation of respiration rate.

The method 250 may also include displaying the determined respiration rate (block 258). The respiration rate may be displayed on the patient monitor 14, the capnograph 18, and/or the multi-parameter monitor 24. In certain embodiments, the respiration rate may be displayed based on a determination that the signal or signal segment does not include one or more features indicative of irregular breathing. In one embodiment, respiration rate may not be displayed based on a determination that the signal or signal segment includes one or more features indicative of irregular breathing. For example, it may be desirable to prevent the display of respiration rate based upon a determination that the respiration rate was calculated using data that may include one or more artifacts that may adversely affect the accuracy of the calculation.

In other embodiments, the method 250 may include providing an indication of irregular breathing (block 260) based upon a determination that one or more features indicative of irregular breathing are present. For example, in certain embodiments, the indication of irregular breathing may be provided instead of displaying the respiration rate. Thus, the method 250 may provide information to the user regarding the absence of the calculated respiration rate. In one embodiment, the absence of the calculated respiration rate may be the indication of irregular breathing. In other embodiments, the indication of irregular breathing may be provided in combination with the displayed respiration rate. In this manner, the indication of irregular breathing may inform the user that the calculated respiration rate may not be accurate as a result of the patient breathing irregularly.

In certain embodiments, providing the indication of irregular breathing may include displaying text, a symbol, graphic, and/or any other suitable display on a display of the patient monitor 14, the capnograph 18, and/or the multi-parameter monitor 24. In some embodiments, providing the indication of irregular breathing may include altering the displayed waveform (e.g., the plethysmographic waveform 134 and/or the carbon dioxide waveform 142). For example, the patient monitor 14, the capnograph 18, and/or the multi-parameter monitor 24 may be configured to remove a portion of the waveform corresponding to the signal segment including the one or more features indicative of irregular breathing, to change the color and/or line quality of the portion of the waveform, to shade the portion of the waveform, to add text and/or a graphic to the portion of the waveform, or any other suitable technique. Further, in some embodiments, providing the indication of irregular breathing may include providing an audible alarm and/or an indicator light via the patient monitor 14, the capnograph 18, and/or the multi-parameter monitor 24.

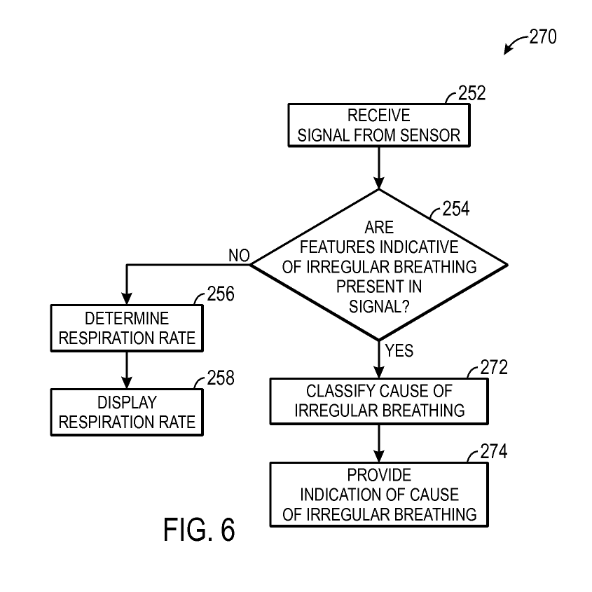

As noted above, the patient monitor 14, the capnograph 18, and/or the multi-parameter monitor 24 may be configured to detect one or more features indicative of irregular breathing and/or to determine the cause of the irregular breathing (e.g., the type of artifact), such as talking, moving, coughing, sneezing, and/or yawning. For example, FIG. 6 illustrates a method 270 for determining the cause of the presence of one or more features indicative of irregular breathing in a waveform (e.g., the plethysmographic waveform 134 and/or the carbon dioxide waveform 142). The method 270 may include receiving one or more signals from one or more sensors (block 252) and determining if one or more features indicative of irregular breathing are present in the one or more waveforms of the one or more received signals (block 254), as described above with respect to FIG. 5. Additionally, as noted above, the method 270 includes determining respiration rate (block 256) and displaying the respiration rate (block 258) in response to a determination that features indicative of irregular breathing are not present in the signal segment.

Further, the method 270 may include classifying (e.g., identifying) the cause of the irregular breathing (block 272). In some embodiments, classifying the cause of the irregular breathing may include identifying one or more features that are indicative of a certain type of irregular breathing, such as talking or motion. For example, classifying the cause of the irregular breathing may include determining a characteristic of the one or more features, and the characteristic may be an association or relationship between a type of feature or a combination of certain features and a type of irregular breathing. As noted above, talking may result in sharp inhalations and/or slow exhalations. Accordingly, detecting such features in the waveform may facilitate the classification of the cause of the irregular breathing as talking. Additionally, in certain embodiments, detecting irregular peak portions (e.g., ripples) in the waveform in the absence of sharp inhalations and/or slow exhalations may indicate that the patient is moving. Accordingly, detecting such features in the waveform may facilitate the classification of the cause of the irregular breathing as motion. In some embodiments, a memory (e.g., the ROM 52 and/or the RAM 54 of the patient monitor 14 and/or the ROM 108 and/or the RAM 110 of the capnograph 18) may be configured to store the characteristics for one or more features indicative of irregular breathing. In one embodiment, the characteristics may be stored as a look-up table. For example, the processor 46 and/or the processor 104 may be configured to access the memory and determine the characteristic of the feature or the features based on the type of feature (e.g., sharp inhalation, slow exhalation, irregular peak portions, etc.) or the combination of features. Furthermore, as noted above, the system 10 may be configured to analyze signals generated by two or more sensors, such as plethysmographic sensors, carbon dioxide sensors, motion sensors, pressure sensors, temperature sensors, and the like, to aid in the identification of the cause of the irregular breathing. For example, in some embodiments, the patient monitor 14, the capnograph 18, and/or the multi-parameter monitor 24 may be configured to compare signals generated by two or more sensors to facilitate the classification of the cause of the irregular breathing.

Additionally, the method 270 may include providing an indication of the cause of the irregular breathing (block 274) based on the classification. As noted above, the respiration rate may be calculated and displayed in response to a determination that one or more features indicative of irregular breathing are not present in the signal or signal segment. However, in other embodiments, the respiration rate may be calculated and displayed regardless of the presence of the one or more features indicative of irregular breathing, and the indication of the cause of the irregular breathing may be provided in combination with the displayed respiration rate. The providing the indication of the cause of irregular breathing may include displaying text (e.g., talking, motion, yawning, sneezing, coughing, and so forth), a symbol, graphic (e.g., an image of talking, motion, yawning, sneezing, coughing, and so forth), and/or any other suitable display that provides an indication of the cause on a display of the patient monitor 14, the capnograph 18, and/or the multi-parameter monitor 24. In some embodiments, providing the indication of irregular breathing may include altering the displayed waveform (e.g., the plethysmographic waveform 134 and/or the carbon dioxide waveform 142). For example, the patient monitor 14, the capnograph 18, and/or the multi-parameter monitor 24 may be configured to remove a portion of the waveform corresponding to the signal segment including the one or more features indicative of irregular breathing, to change the color and/or line quality of the portion of the waveform, to shade the portion of the waveform, to add text and/or a graphic to the portion of the waveform, or any other suitable technique. Further, in some embodiments, providing the indication of irregular breathing may include providing an audible alarm and/or an indicator light via the patient monitor 14, the capnograph 18, and/or the multi-parameter monitor 24.

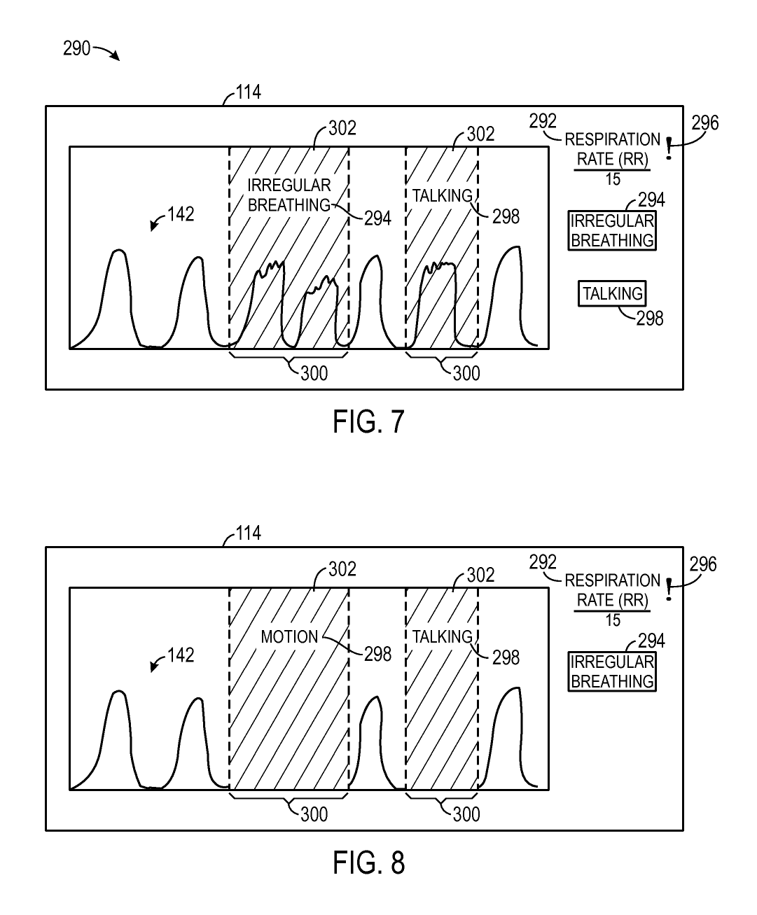

As noted above, the various indications of irregular breathing and the indications of the cause of irregular breathing may be provided using the patient monitor 14, the capnograph 18, and/or the multi-parameter monitor 24. Accordingly, while the embodiments described below with respect to FIGS. 7 and 8 are described in the context of the display 114 of the capnograph 18, it should be noted that the embodiments may be displayed on any suitable display, such as the display 58 of the patient monitor 14 or a display of the multi-parameter monitor 24. Furthermore, while the embodiments described below with respect to FIGS. 7 and 8 are described in the context of the carbon dioxide waveform 142, it should be noted that the present techniques may be implemented using the plethysmographic waveform 134, any other suitable waveform or signal, or a combination thereof.

For example, FIG. 7 is an illustration 290 of the display 114 of the capnograph 18 that may display the carbon dioxide waveform 142, a calculated value of respiration rate 292, and any other suitable waveforms, physiological parameters, and/or user indications. As illustrated, the carbon dioxide waveform 142 includes periods of irregular breathing. In response to detecting the periods of irregular breathing, the processor 104 may be configured to cause the display to display an indication of irregular breathing 294. The indication of irregular breathing 294 may include a textual indication, such as “irregular breathing” or any other text suitable for conveying to a caregiver that the patient may be breathing irregularly and/or that the accuracy of the calculated respiration rate may be adversely affected. As illustrated, the indication of irregular breathing 294 may be displayed below the value of respiration rate 292 or in any other suitable location. Additionally, the indication of irregular breathing 294 may be displayed as a tab, a banner, a dialog box, or any other suitable type of display. Additionally or alternatively, the indication of irregular breathing 294 may include a symbol 296, such as an exclamation point, an asterisk, a star, or a stop sign. In other embodiments, the processor 104 may be configured to alter the color, size, font, and/or shading of the value of respiration rate 292 in response to a determination that the patient is breathing irregularly. Additionally, in embodiments in which the processor 104 is configured to classify the cause of the irregular breathing, the indication of irregular breathing 294 may include an indication of the cause of the irregular breathing 298, which may be a textual indication, such as “talking” or any other text suitable for conveying the determined cause of the irregular breathing, a symbol, a graphic, or the like.

Additionally, the processor 104 may be configured to alter the carbon dioxide waveform 142 to provide the indication of irregular breathing. In certain embodiments, the processor 104 may be configured to alter the carbon dioxide waveform 142 to identify the portions of the carbon dioxide waveform 142 corresponding to periods of irregular breathing 300. For example, as illustrated in FIG. 7, the processor 104 may be configured to provide a shaded region 302 over portions of the carbon dioxide waveform 142 that the processor 104 has determined correspond to irregular breathing. However, it should be noted that other techniques may be used to identify the portions of the waveform, such as altering the color, thickness, and/or line quality of the waveform. Further, the processor 104 may be configured to cause the display of the indication of irregular breathing 294 in the shaded region 302 or proximate to the shaded region 302. Additionally, the processor 104 may be configured to cause the display of the indication of the cause of the irregular breathing 298 in the shaded region 302 or proximate to the shaded region 302.

In other embodiments, the processor 104 may be configured to remove portions of the carbon dioxide waveform 142 corresponding to periods of irregular breathing. For example, as illustrated in FIG. 8, the processor 104 may omit the periods of irregular breathing 300 from the displayed carbon dioxide waveform 142. The omitted periods of irregular breathing 300 may be shaded regions 302, as described above with respect to FIG. 7. In other embodiments, the omitted periods of irregular breathing 300 may not be shaded. In some embodiments, the processor 104 may cause the display of the indication of irregular breathing 294 in the shaded regions 302 and/or the display of the indication of the cause of the irregular breathing 298. For example, as illustrated, the carbon dioxide waveform 142 includes a first indication of the irregular breathing 298 that identifies the cause of a first period of irregular breathing 300 as motion and includes a second indication of irregular breathing 298 that identifies the cause of a second period of irregular breathing 300 as talking.

The techniques provided herein have been illustrated with reference to the monitoring of a physiological signal (which may be a photoplethysmographic signal or an end-tidal carbon dioxide signal); however, it will be understood that the disclosure is not limited to monitoring physiological signals and is usefully applied within a number of signal monitoring settings. Those skilled in the art will recognize that the present disclosure has wide applicability to other signals including, but not limited to, other biosignals (e.g., electrocardiogram, electroencephalogram, electrogastrogram, electromyogram, heart rate signals, pathological sounds, ultrasound, or any other suitable biosignal), any other suitable signal, and/or any combination thereof.

While the disclosure may be susceptible to various modifications and alternative forms, specific embodiments have been shown by way of example in the drawings and will be described in detail herein. However, it should be understood that the disclosure is not intended to be limited to the particular forms disclosed. Rather, the disclosure is to cover all modifications, equivalents and alternatives falling within the spirit and scope of the disclosure as defined by the following appended claims. Further, it should be understood that elements of the disclosed embodiments may be combined or exchanged with one another.

What is claimed is:

1. A method, comprising:
receiving, via a monitor, a physiological signal from a carbon dioxide sensor;
determining a respiration rate, via the monitor, from the physiological signal;
displaying the respiration rate on a display screen;
screening, via the monitor, the physiological signal for a characteristic indicative of talking;
recognizing, via the monitor, that the characteristic indicative of talking is present in the physiological signal;
in response to recognizing that the characteristic indicative of talking is present, removing the respiration rate from the display screen; and
displaying a talking indicator on the display screen.

2. The method of claim 1, wherein screening the physiological signal for the characteristic indicative of talking comprises identifying, via the monitor, two or more breath periods in a segment of the physiological signal.

3. The method of claim 2, wherein recognizing that the characteristic indicative of talking is present comprises determining, via the monitor, a spread of a distribution of the two or more breath periods and determining, via the monitor, that the spread of the distribution is greater than a predetermined threshold.

4. The method of claim 1, wherein the characteristic indicative of talking comprises irregular periodicity of breath periods, asymmetric breath periods, short inhalations relative to exhalations, sharp inhalations, or irregular peaks of the physiological signal.

5. The method of claim 1, wherein the physiological signal comprises an end-tidal carbon dioxide signal.

6. The method of claim 1, wherein the talking indicator comprises text, a symbol, or a graphic.

7. The method of claim 1, wherein the characteristic indicative of talking comprises a sharp inhalation, a slow exhalation, or both.

8. The method of claim 1, further comprising altering a display of a waveform of the physiological signal along segments in which the characteristic indicative of talking is present.

9. The method of claim 8, wherein altering the display of the waveform comprises changing a color, shading, or line quality.

10. The method of claim 8, wherein altering the display of the waveform comprises shading the display along the segments.

11. The method of claim 1, comprising receiving, via the monitor, a second physiological signal from a plethysmographic sensor, and wherein the screening comprises screening both the first and the second physiological signals for respective characteristics indicative of talking.

12. A method for identifying talking interruptions in a respiratory signal, comprising:
receiving, via a monitor, a physiological signal from a carbon dioxide sensor;
determining a respiration rate, via the monitor, from the physiological signal;
displaying the respiration rate on a display screen;
displaying on the display screen a waveform of respiration over time based on the physiological signal;
screening, via the monitor, the physiological signal for a characteristic indicative of talking;
recognizing, via the monitor, that the characteristic indicative of talking is present in the physiological signal;
in response to recognizing that the characteristic indicative of talking is present, displaying a talking indicator on the display screen; and
altering the display of the waveform along segments in which the characteristic indicative of talking is present.

13. The method of claim 12, wherein the talking indicator comprises text, a symbol, or a graphic.

14. The method of claim 12, further comprising removing the respiration rate from the display screen upon recognizing that the characteristic indicative of talking is present in the physiological signal.

15. The method of claim 12, wherein the characteristic indicative of talking comprises short inhalations relative to exhalations.

16. The method of claim 12, wherein the characteristic indicative of talking comprises sharp inhalations, slow exhalations, irregular peaks of the physiological signal, or a combination thereof.

17. A method for identifying talking interruptions in a respiratory signal, comprising:
receiving, via a monitor, a physiological signal from a carbon dioxide sensor;
determining a respiration rate, via the monitor, from the physiological signal;
displaying the respiration rate on a display screen;
detecting, via the monitor, sharp inhalations relative to slow exhalations in the physiological signal; and
in response to detecting the sharp inhalations relative to the slow exhalations, displaying a talking indicator on the display screen.

18. The method of claim 17, further comprising removing the respiration rate from the display screen, upon detecting the sharp inhalations relative to the slow exhalations.

19. The method of claim 17, wherein the talking indicator comprises text, a symbol, or a graphic.

20. The method of claim 17, further comprising altering a display of a waveform of the physiological signal along segments in which the sharp inhalations relative to the slow exhalations are present.
